(19) United States
(12) Patent Application Publication
(10) Pub. No.: US 2012/0238865 A1
(43) Pub. Date: Sep. 20, 2012

(54) Method for Measuring the Physiological Parameters of Brain Interstitial Fluid and Brain Extracellular Space

(76) Inventor: Hongbin Han, Beijing (CN)

(21) Appl. No.: 13/511,681

(22) PCT Filed: Dec. 7, 2009
(86) PCT No.: PCT/CN2009/075360

§ 371 Date: May 25, 2012

(51) Int. Cl. A61B 5/055

(52) U.S. Cl. 600/420

(57) ABSTRACT

The invention would provide a method of measuring the physiological parameters of brain interstitial fluid (ISF) and brain extracellular space (ECS). The method is available to obtain the extract physiological parameters of the substances distributing, diffusing and eliminating in the brain ECS. The details are as follows: The head of the object was settled in magnetic resonance imaging (MRI)system; the MRI contrast agent was injected into brain ISF; the signal intensity changed by the MRI contrast agents was detected on MR images; the distribution of the contrast agents in the brain can be quantitatively analyzed by the signal intensity the concentration of contrast agents and its time-dependent change of the different brain regions can be acquired. The invention can be feasible to quantify the indexes of the brain ISF distribution, fluidity and dissemination in the cerebral ECS by the signal intensity on MR images. The indexes including the anatomical and physiological parameters of the brain ISF and cerebral ECS can also be extracted by the invention.

### FIELD OF THE INVENTION

The present invention relates to a method for measuring the physiological parameters of brain interstitial fluid and brain extracellular space. In particular, it related to a method for assessing the physiological parameters of brain interstitial fluid and brain extracellular space by measuring the concentration change of the contrast agents in the brain interstitial fluid in the brain extracellular space to detecting the distribution, diffusion and elimination of the contrast agents.

### BACKGROUND OF THE INVENTION

The brain interstitial fluid (ISF) is a solution found in the brain extracellular space (ECS). The brain ECS, also known as tissue channel, refers to the irregular, interconnected and narrow space which in the interstitial tissue and between cells membranes. According to Nicholson, brain ECS, brain ISF and extracellular matrix (ECM) constitute the brain extracellular microenvironment (BEM), which plays a important role in maintaining the stability of the electronic signals among the brain cells, forming the transport channels between the cell and the blood, and remodeling the neural synaptic. Therefore, it's needed to study the physiological parameters of brain ISF and ECS and discover the rules of the neural activity.

In the micro circulation field, however, there had been an argument about the anatomy of the brain ECS, as well as the quantitative measurement and analysis of the mobility physiology index of the brain ISF. In the beginning, the brain ECS was almost invisible under the electron microscopy because of the limited technology of the specimen making and post-processing. Later, the brain ECS was discovered due to exploration and application of the new technology. By RTI-TMA technology (a kind of real-time iontophoresis technology which uses NH4+TMA as the tracer) we learn that the brain ECS accounts for about 20% volume of the brain and the width of the space is 38 nm to 64 nm and has variability. In the current mature measuring methods of brain ECS, real-time iontophoresis (RTI), real-time pressure ejection(RTP), radioactive tracer method and integrative optical imaging(IOI) are most commonly used.

In RTI and RTP, two tiny ion selective electrodes are inserted into brain (one releases ions and the other accepts them) to monitor the diffusion of ions between two points in a certain region of the brain. Basing on the movement of the ions in the certain area, the brain ECS structure is available. But these two methods can only measure the diffusion of limited ions such as potassium ion (K+) and calcium ion (Ca2+) in brain ECS in a small fixed region(e.g. 60 μm to100 μm).

In radioactive tracer method, through injecting the radioactive substance into the brain and incising the different sections of the brain at different time to measure the radioactive dose, the diffusion data is available. But this method must kill an animal in each measuring time, and only applies to the big brain such as a dog brain and a monkey brain.

Injecting the fluorescent substance into the brain and recording the changes of the fluorescence intensity of the fluorescent substance in real-time by fluorescent microscope and high resolution charge coupled device camera (CCD camera); integrative optical imaging (IOI) can analyze the diffusion of the substance. However, since the penetration of fluorescein is relatively weak, we can only use this method to monitor fluorescent changes within a region 200 μm far from the brain surface.

Among the above methods, only MI provides the image of the brain superficial tissue when monitoring the diffusion of the substance, and the rest can not realize the visualization measurement. All of these methods lack effective means for measuring the physiological parameters of the brain ISF and ECS, such as flow rate, resistance, pressure and so on.

The magnetic resonance imaging (MRI) is the most commonly imaging detection technique in recent years. It is used to observe the anatomical structure and physiological function of people or animals. MRI is a real time, visible and noninvasive technique which can be used for living body. The contrast agents have further enhanced the application of the MRI.

At present, there are two kinds of contrast agents used in MRI one is T1 positive contrast agent such as GD-DTPA, and the other is T2 negative contrast agent such as iron nanoparticles.

Some contrast agents can also act as physical tracer in MRI. A preliminary research has started to use the iron nanoparticles as a MRI tracer to trace the clearance of the metabolites of ISF. The studies shows that the injected iron nanoparticles are get into the cervical lymph node via lymph of the nasal mucosa and then cleared out of the brain.

However, the studies also have demonstrated that the spread of the contrast agents in brain is unclearly due to image distortion and widespread signal loss in the result of interference on gradient magnetic field caused by iron nanoparticles. Therefore, accurate observation and quantitative analysis of the brain ISF and ECS is unrealizable by this method.

Some studies the measurement of the brain ECS via diffusion weighted imaging (DWI). DWI is one of the MRI techniques measuring the apparent diffusion coefficient (ADC) and anisotropy fraction (AC) of the water molecules or other molecules in body. This method is bases on the theory that molecular diffusion makes the MRI image signal change. If a sensitive magnetic gradient field is added in one direction, the more obvious the diffusion in this direction of the organ, the lower the collected MRI signal is, and vice versa. The ADC can be concluded from different MRI signal strength in different diffusion sensitive magnetic gradient. The diffusion tensor factor, such as FA, can be obtained by adding sensitive magnetic gradient field in six different directions. When the measured molecules only exist in brain ECS, neither enter in the cells nor reverse transport through the blood-brain-barrier, the ADC and FA can reflect the shape of the ECS indirectly. If the molecules diffusion is limited due to the straitness of the brain space, the ADC will decrease and the FA will change according to the tortuosity of ECS. However, the clinical DWI often chooses water molecules as the tracer and the diffusion of the water molecules not only exists in brain ECS but also in cells. As a result, the ADC value contains the result in these two parts. Therefore, this method can not accurately describe the diffusion of the brain ECS.

Some studies choose TMA as the RF-excited object in MRI. The ADCTMA (e.g. the diffusion coefficient of the brain ECS) is obtained via magnetic resonance spectroscopy (MRS). These studies show that the ADCTMA is far below the diffusion coefficient in RTI-TMA and only a quarter of the latter. Furthermore, the resolution of the MRS is so low (0.5*0.5*0.5 cm3) that the diffusion of contrast agents within a specific region of brain which is smaller than 0.2*0.2*0.2 cm3 is unavailable. Therefore, position measurement of the brain ECS accurately by this method is unreachable.

### SUMMARY OF THE INVENTION

The present invention provides a method for measuring the physiological parameters of brain ISF and brain ECS. Using the change of the MRI signal intensity (SI) caused by the diffusion of the contrast agents in brain, the present invention can calculate the diffusion properties of brain ECS, thus obtain the anatomical structure and the physiological parameters of the brain ECS as well as the location change of the contrast agents due to the diffusion and the process of elimination.

The present invention provides a method for measuring the physiological parameters of brain interstitial fluid and brain extracellular space. The method comprises: putting head of the object into magnetic resonance imaging environment; injecting contrast agents of magnetic resonance imaging into brain interstitial fluid in brain extracellular space of the object; measuring signal intensity of image arising from the contrast agents in the magnetic resonance imaging system; and determining concentration, rate of change of concentration and distribution of the contrast agents depending on the signal intensity.

Certain exemplary embodiments can provide a method, in which the contrast agents are injected into the brain interstitial fluid in the brain extracellular space of the object by centesis.

Certain exemplary embodiments can provide a method, in which the magnetic resonance imaging is scan by a three dimensional gradient echo T1 weighted sequence.

Certain exemplary embodiments can provide a method, in which the contrast agents are Gd-DTPA.

Certain exemplary embodiments can provide a method, in which the concentration range of the contrast agents is within 0 to 1 mM the relationship between the signal intensity of the contrast agents in magnetic resonance imaging environment and the concentration thereof at a certain region of the brain of the object is as follows,

$${C}_{Gd} = \frac{SI - B}{K} ,$$

wherein:
SI is the signal intensity of the contrast agents at the certain site in the magnetic resonance imaging environment,
CGd is the concentration of the contrast agents at the certain site,
K is a constant, which is in the range of 300-3000, and
B is a constant, which is in the range of 20-200.

Utilizing magnetic resonance imaging (MRI) technique and via the changes of MRI signal intensity (SI) caused by the diffusion and elimination of the contrast agents in the brain, the present invention can obtain the anatomical structure and the physiological parameters of the brain ECS.

In particular, by observing and measuring the parameters of diffusion and the elimination of the MRI contrast agents in ISF of the brain ECS, the present invention can accurate observation and quantitative analysis of the anatomical structure and the physiological parameters of the brain ECS in different brain area in vivo. The present invention also can accurately estimate the diffusion and the elimination of the matter having the same molecular weight and polarity with contrast agents in brain ECS. It can provide useful information for the research of cerebral microcirculation.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 shows a relation curve of the SI and the concentration of the contrast agents in the brain ECS in a MRI environment.

FIG. 2 shows a linear relation between the SI and the concentration in the curve shown in FIG. 1 when the concentration of the contrast agents is in the range of 0-0.1 mM.

FIG. 3 shows a fitting curve of SI and the concentration of the contrast agents in another MRI environment.

FIG. 4 shows a schematic view of an apparatus used in present method for measuring the physiological parameters of brain ISF and ECS.

FIG. 5 shows a schematic view showing the injection site of the contrast agents in brain ECS.

FIG. 6 shows a curve of SI increment vs. time of the contrast agents in the same direction as in FIG. 5 and different site as in FIG. 5.

FIG. 7a to FIG. 7e show MRI images of the rat brain at different times after injecting the contrast agents.

FIG. 8a to FIG. 8d show MRI diffusion images of the time-dependent of the contrast agents after injecting into the white matter fiber areas of the rat brain.

### DETAILED DESCRIPTION OF THE EMBODIMENTS

The preferred embodiments of the present invention are described below with reference to the accompanying drawings.

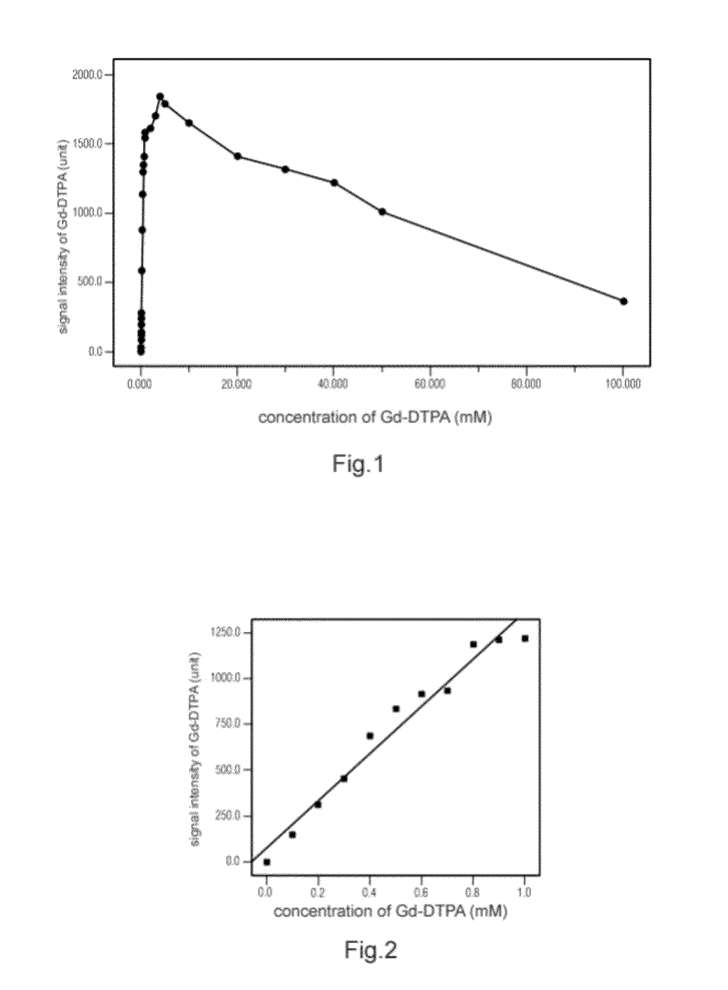

FIG. 1 is shows a relation curve of the SI and the concentration of the contrast agents in the brain ECS in a MRI environment. It shows the relationship between the SI and the concentration of the contrast agents Gd-DTPA in agarose at 37. According to the curve, when the concentration of the contrast agents is in the range of 0-5 mM, the SI increased with the concentration. The concentration is related to the SI. In the curve, the abscissa is the concentration of the contrast agent which reflects the mobility of the ISF in brain ECS and the ordinate is the SI of MRI. It is known by the persons skilled in art that the SI here may be the directly SI or the result which is calculated indirectly from the SI such as T1. In the exemplary embodiment, the contrast agents are Gd-DPTA. Other non-ferromagnetic, non-neurotoxicity and extracellular contrast agents, such as T1 positive MRI contrast agent, also can be used here.

In the exemplary embodiment shown in FIG. 1, although the relation curve between SI and the concentration of Gd-DTPA is measured in agarose, the persons skilled in art understand that that the agarose and the cerebral tissue both allow Gd-DTPA to be diffused and are diffusion media to the Gd-DTPA. The essence of Gd-DTPA diffused in these two media is that Gd-DTPA is diffused in the interval thereof. The different is that the interval of agarose is full of water and the interval of cerebral tissue is full of CSF which contains water primarily, some ions and few proteins. Although the ions and the protein may affect the SI result of Gd-DTPA, there is still a linear relationship between the SI and the concentration of the contrast agents.

FIG. 2 shows a linear relation between the SI and the concentration in the curve shown in FIG. 1 when the concentration of the contrast agents is in the range of 0-0.1 mM. According to the curve shown in FIG. 2, the liner equation of the curve is obtained as below

$${C}_{Gd} = \frac{SI - B}{K} \qquad ( 1 )$$

wherein:
SI is the signal intensity of the contrast agents at a certain site in the MRI environment,
CGd is the concentration of Gd-DTPA at the certain site,
K is a constant and represents the slope of this curve,
B is a constant and represents the SI without injection of Gd-DTPA.

Table 1 below displays four experimental groups and shows that the values of K and B are affected by the factors such as magnetic field intensity, MRI sequence, kind of contrast agents and the concentration range of contrast agents. In the four experiments, injecting contrast agents with different concentration range are into agarose, the distribution of SI of the MRI is measured in different magnetic field intensity or different MRI sequence. When quantitative measuring the contrast agents via the SI, the value of K and B can be determined via linear fitting according to the experimental conditions,

TABLE 1

| | Experi- | Experi- | Experi- | Experi- |
|---|---|---|---|---|
| | ment 1 | ment 2 | ment 3 | ment 4 |
| Magnetic field | 1.5T | 3T | 3T | 3T |
| intensity | | | | |
| MRI sequence | FLASH 2D | FLASH 2D | 3D | 3D |
| | | | MP-RAGE | MP-RAGE |
| Contrast agent | Gd-DTPA | Gd-DTPA | Gd-DTPA | Gd-DTPA |
| Concentration | 0-1.2 mM | 0-1.2 mM | 0-0.5 mM | 0-1 mM |
| range | | | | |
| Value of K | 337.434 | 247.834 | 1913.686 | 1174.5 |
| Value of B | 184.344 | 45.323 | 29.662 | 197.732 |

When the linear fitting was performed at different concentration ranges, the values of K and B varies. Experiments and calculations have demonstrated that the value of K is in the range of 300 to 3000 and the value of B is in the range of 20 to 200. The narrower the concentration range is, the larger the value of K and the smaller the value of B will be, vice versa.

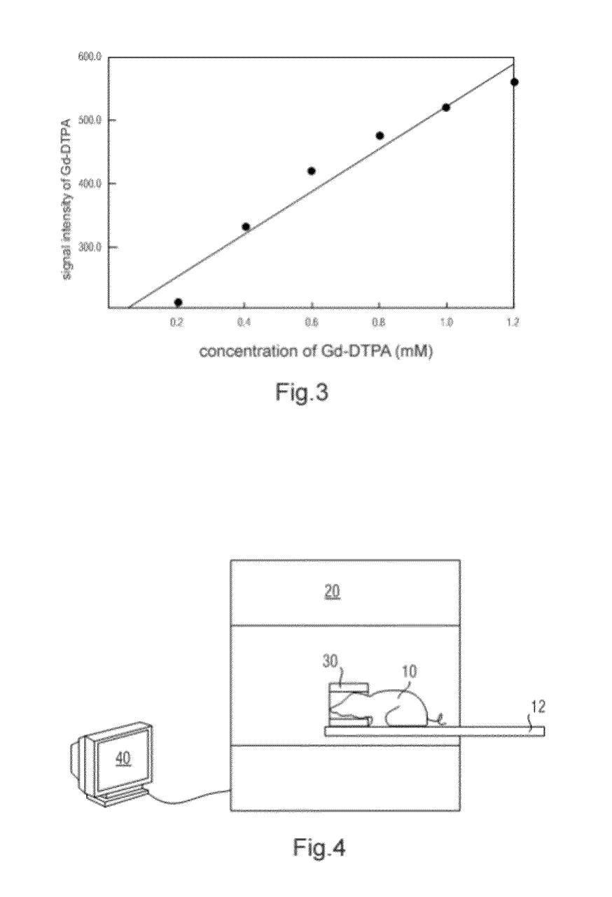

FIG. 3 is a fitting curve of the SI and the concentration of the contrast agents corresponding to the Experiment 1 shown in Table 1. It is the measurement result under the circumstances that the magnetic field intensity is 1.5T, the MRI sequence is FLASH 2D, the contrast agent is Gd-DTPA and the concentration range is 0 to 1.2 mM. According to the fitting curve, the value of K is 337.434 and the value of B is 184.344.

FIG. 4 shows a schematic view of an apparatus used in present method for measuring the physiological parameters of brain ISF and ECS. The apparatus includes an imaging device 20 and a controlling device 40. The imaging device can be CT, MRI and so on. The imaging device 20 is connected with the controlling device 40.

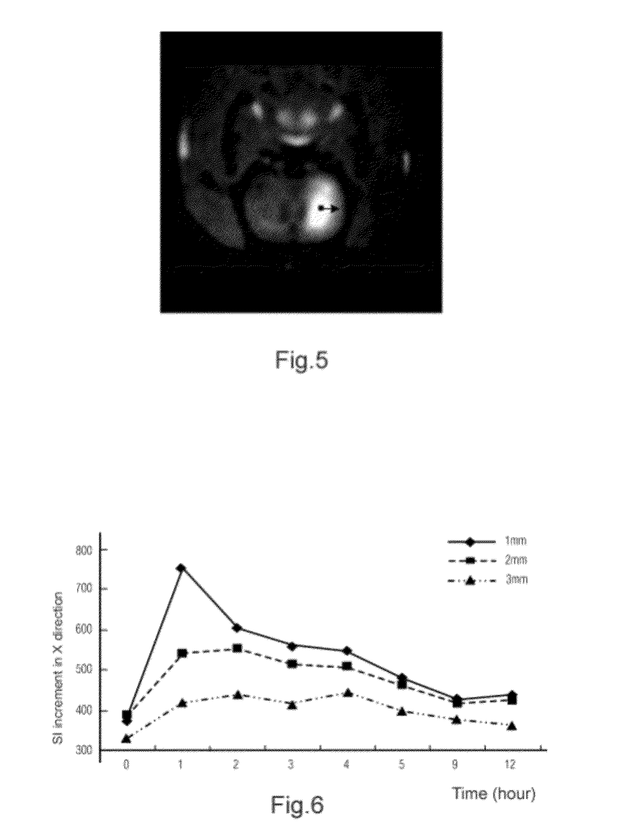

In this exemplary embodiment, the rat 10 is anesthetized and incised in the scalp along the sagittal suture. The periosteum was separated and the bregma was exposed. 1 μL Gd-DTPA with concentration range of 5 to 25 mM is injected into the brain ECS of the rat caudate nucleus at the rate of 0.1 μL/min. Basing on The Rat Brain in Stereotactic Coordinates (3rd Edition, People Health Publishing House, 2005, the caudate nucleus is 1.0 mm away from the front of the bregma anterior, 3.0 mm away from the left and 4.5 mm away from the depth/vertical. In FIG. 5, the round spot shows the injection site of Gd-DTPA in the caudate nucleus of the rat brain, and the arrow represents the X direction, e.g., the direction along the center point line of rat external auditory canal.

The microsyringe is slowly removed 5 minutes after injecting. In FIG. 4, the rat narcotized is placed in the wrist coil 30 in the prone position and sent into the imaging device 20 with the examination couch 12. A T1 weighted sequence is used for the MRI scanning MR images from the imaging device 20 are processed in the image-processing unit of the controlling device 40.

FIG. 6 is a curve of SI increment (ASI) vs. time of the contrast agents along X direction of FIG. 5 at the spots respectively 1 mm, 2 mm, and 3 mm away from the injection site. The increment of SI reflected the concentration increment of the contrast agents. In FIG. 6, the peak of ASI appears in the region which is 1 mm away from the injection site about 1 h after injection, and then value of ASI slowly falls down. At the spots 2 mm and 3 mm away from the injection site, which are located in the area of cortex, the value of ASI (i.e. the concentration increment of the contrast agents) first slowly increases then slowly decreases.

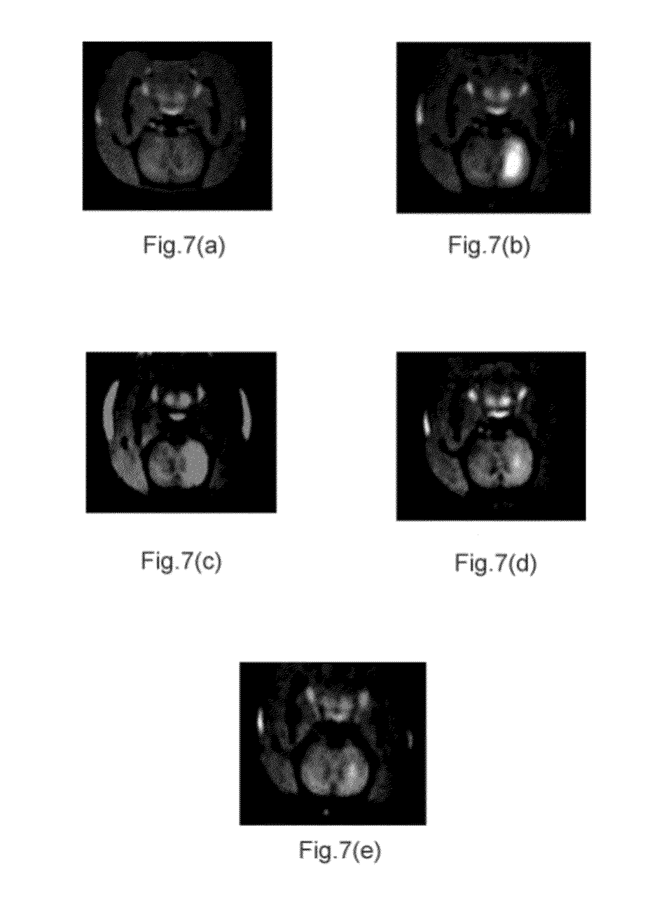

FIG. 7a to FIG. 7e are MRI images of the rat brain at different time after injecting of the contrast agents by the apparatus shown in FIG. 4. FIG. 7a shows the MRI image of the central region of the rat caudate nucleus before the injection of contrast agents. FIG. 7b shows the MRI image of the central region of the rat caudate nucleus 1 hour after the injection of contrast agents. FIG. 7c shows the MRI image of the central region of rat caudate nucleus 5 hours after the injection of Gd-DTPA. FIG. 7d shows of the central region of the MRI image of the rat caudate nucleus 6 hours after the injection of Gd-DTPA. FIG. 7e is the MRI image of the central region of rat caudate nucleus 10 hours after the injection of Gd-DTPA.

By the similar methods, the MRI images in three orthogonal directions can be obtained. Depending on the result of the MRI at different time point after injection, the curve in three orthogonal directions, as the curve shown in FIG. 6, can be obtained.

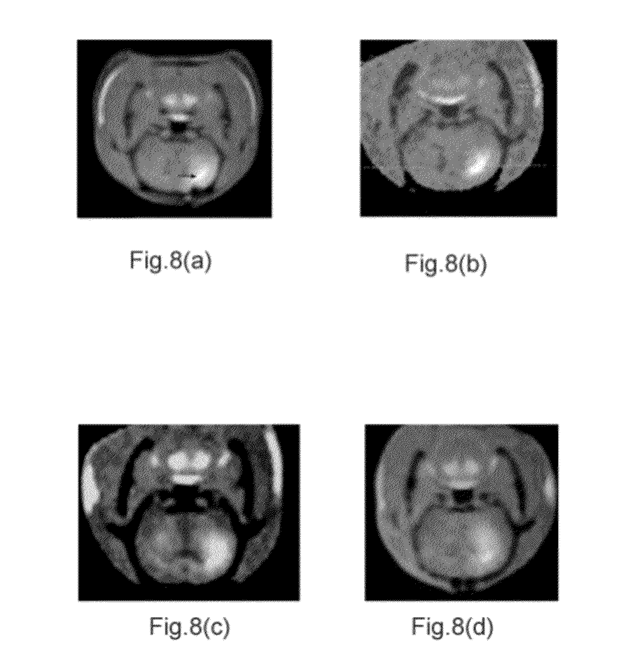

In the same way, the values of MR signal intensity at different time on different site of the brain can be measured when the quality of the brain ECS is measured. FIG. 8a to FIG. 8d show MRI images after injecting the contrast agents into the white matter fiber areas of the rat brain. FIG. 8a shows the MRI image of the white matter fiber areas of the rat brain before the injection of contrast agents. FIG. 8b shows the MRI image of the white matter fiber areas of the rat brain 1 hour after the injection of contrast agents. FIG. 8c shows the MRI image of the white matter fiber areas of the rat brain 3 hours after the injection of Gd-DTPA. FIG. 8d shows the MRI image of the white matter fiber areas of the rat brain 6 hours after the injection of Gd-DTPA.

FIG. 7a to FIG. 7e and FIG. 8a to FIG. 8d indicate that the method shown in this exemplary embodiment can clearly show and fitting measure the change of the SI caused by Gd-DTPA in different areas of brain and reflect the change and the change rate of the concentration of Gd-DTPA. This method can not only display and quantitatively analyze the diffusion, the flow and the elimination of Gd-DTPA as well as the physiological process of the same or similar molecular to Gd-DTPA in the brain ECS.

In the present invention, the concentration of Gd-DTPA can be calculated by measuring the SI of Gd-DTPA in a certain site at a certain time. Through the dynamic monitor of the imaging device and the real-time analysis of the control device, the distribution of diffused Gd-DTPA in the brain and the elimination process of Gd-DTPA can be obtained at any time. By Formula 1, the concentration of Gd-DTPA in ECS is known and other physiological parameters of ECS in each pixel, such as tortuosity X, volume ratio a of the brain ECS to brain tissue, diffusion coefficient D and so on, can be figured out according to the known methods. Thus flow properties of ECS can be measured according to the diffusion of contrast agent. The size of pixel is dependent upon the performance of MRI device. Generally, by the existing technology the smallest size of pixel is 0.01 mm to 0.1 mm. In an exemplary embodiment of the present invention, the pixel is 0.5*0.5*0.5 mm.

For example, the following Nicholson formula can be used to calculate the flow properties of ECS:

$$\frac{\partial C}{\partial t} = \frac{D}{{λ}^{2}} {∇}^{2} C + \frac{Q}{α} - v \cdot ∇ C - \frac{f ( C )}{α} \qquad ( 2 )$$

In formula 2:
C is the concentration of Gd-DTPA in the calculated site;
v is the injection speed of Gd-DTPA;
λ is the tortuosity of ECS;
α is the volume fraction of the brain ECS to brain tissue, which is available via formula 3 shown below:

$$α = \frac{Vecs}{Vtissue} α = \frac{{V}_{ECS}}{{V}_{Tissue}} \qquad ( 3 )$$

In formula 3, Vtissue is the volume of brain tissue and Vecs is the volume of brain ECS. ECS accounts for 15-30%, average 20% volume of the brain tissue of a normal adult. The percentage is decreased to 5% when the cerebral ischemia happens.

The diffusion coefficient D represents the diffusion mode of molecule in infinite medium such as diluted agarose. The measuring method includes: injecting 24, Gd-DTPA (25mM) into 1% agarose gel by three-dimensional positioning technique; scanning via T1 weighted sequence 30 min (t1) and 60min (t2) after injecting; measuring the diffusion area s1 and s2 of contrast agents at the surface perpendicular to the direction of the injecting needle with software, and calculating D according to formula 4 shown below:

$$D = \frac{s 2 - s 1}{t 2 - t 1} \qquad ( 4 )$$

The diffusion coefficient of molecular in medium with certain tortuosity is effective diffusion coefficient D*, and

$${D}^{*} = \frac{D}{{λ}^{2}} ;$$

Diffusion source Q is the amount of contrast agent released into ECS per unit time, and is dependent upon the speed of injection. For example, if the contrast agent is injected at a speed of 0.0501 sec, the value of Q is

0.05 μL/sec. Therefore,

$$\frac{Q}{α}$$

represents the volume of molecular which is released into ECS.

Concentration gradient ∇C is the concentration gradient inducing from the flowing of liquid v·∇c represents the effect caused by bulk flow. If the distance between the two measuring points is short, the effect of the bulk flow can be ignored.

The clearance rate f(c) represents the loss of substance, i.e., the proportion of molecules which pass through the blood-brain barrier (BBB) and enter directly or binding with the receptors. f(c) is the function of volume fraction a and the solution's concentration C and represents the elimination of the solution injected into ECS, e.g., the solution entering the cells, passing through the BBB, degraded by enzyme or lost in other process. The clearance rate can be calculated according to formula 5 shown below.

f(c)=k′·α·c (5)

wherein, k′ is a constant of the clearance rate.

By substituting formula 5 into formula 2, we can get formula 6 shown below:

$$\frac{\partial C}{\partial t} = \frac{D}{{λ}^{2}} {∇}^{2} C + \frac{Q}{α} - v \cdot ∇ C - {k}^{′} C \qquad ( 6 )$$

The properties of molecular diffusion in ECS can be obtained by injecting Gd-DTPA with a concentration of 5 to 25 mM into the targeted brain region at a constant rate, scanning with T1 weighted imaging sequence by using the apparatus shown in FIG. 4, and calculating the tortuosity (λ), volume fraction of ECS (α), diffusion coefficient (D) and the rate constant (k′) basing on formula 3 to 5.

According to the present invention, we can choose and analyze a certain tissue region with the spatial resolution is 0.1×0.1×0.1 cm3 and analyze it separately. The diffusion of the contrast agents in the whole brain are visible and the three dimensional anatomical structural information is available. Furthermore, the measurement can be done whether in vivo or vitro.

The present invention provides the method for measuring the physiological parameters of brain ISF and brain ECS. It realizes a visualized, real-time measurement in vivo in whole brain ECS. Therefore, the structure of ECS and the physiological parameters of ISF fluidity can be measured accurately, which is helpful in the research of cerebral microcirculation, pharmacokinetic and so on.

As can be understood, above detailed illustration is not used to limit the scope of the invention. The invention is defined by the appended claims.

1. A method comprising:
via a magnetic imaging system, measuring a signal intensity of an image from the one or more contrast agents injected in a brain interstitial fluid in a brain extracellular space of an object;
determining concentration, change rate of concentration, and distribution of the one or more contrast agents basing on the signal intensity.

2. The method according to claim 1, wherein the one or more contrast agents are injected into the brain interstitial fluid in the brain extracellular space of the object by centesis.

3. The method according to claim 1, wherein the magnetic resonance imaging is scanned by a three dimensional gradient echo T1 weighted sequence.

4. The method according to claim 1, wherein the one or more contrast agents comprise Gd-DTPA.

5. The method according to claim 41, wherein, when concentration range of the one or more contrast agents is within 0 to 1 mM, a relationship between the signal intensity of the one or more contrast agents in the magnetic resonance imaging system and the concentration thereof at a predetermined position of the brain of the object is as follows:
$${C}_{Gd} = \frac{SI - B}{K} ,$$
wherein:
SI is the signal intensity of the one or more contrast agents at the predetermined site in the magnetic resonance imaging system,
C_{Gd} is the concentration of the one or more contrast agents at the predetermined site,
K is a constant, ranging from 300-3000, and
B is a constant, ranging from 20-200.
